(19) United States
(12) Patent Application Publication
(10) Pub. No.: US 2018/0296404 A1
(43) Pub. Date: Oct. 18, 2018

(54) MENSTRUAL TAMPON CASE

(71) Applicant: Pavel SEKANINA, Mladéjov na Moravé (CZ)

(72) Inventor: Pavel SEKANINA, Mladéjov na Moravé (CZ)

(21) Appl. No.: 15/767,655

(22) PCT Filed: Aug. 23, 2016
(86) PCT No.: PCT/CZ2016/000096

§ 371 Date: Apr. 12, 2018

(30) Foreign Application Priority Data
Oct. 13, 2015 (CZ) PV 2015-725

(51) Int. Cl. A61F 15/00; A61F 13/20; A61F 13/551

(52) U.S. Cl. CPC A61F 15/003; A61F 2013/55195; A61F 13/55175; A61F 13/20

(57) ABSTRACT

Problem to be solved: protecting a menstrual tampon (2) from mechanical damage, from contamination and moisture, and the problem with carrying it discreetly. Solution to the problem: a case (1) for tampons (2) consisting of a rigid body, inside of which there is stored at least a single row of tampons (2) arranged in parallel, provided at the bottom closed end (4) with a dispensing means acting on the outer tampon (2) of the row of tampons (2) and is provided at the open top end (5) with a holder (6) for retaining the tampon (2) in the top end (5) for easy and gradual removal.

### FIELD OF THE INVENTION

The invention relates to a case for holding menstrual tampons that women have prepared with them during menstruation.

### PRIOR ART

There is currently a wide range of sanitary aids to assist women in the capture of menstrual blood during menstruation. One of these sanitary aids is the menstrual tampon.

The menstrual tampon is a disposable sanitary aid shaped like a cylinder, one end of which is rounded for easy insertion while the second end is provided with a string for its removal. The menstrual tampon is usually made from a mixture of silk and cotton. This material has a high absorption capacity and is harmless to the body. The disadvantages of this material and of tampons in general lie in the fact that the material, thus the tampons themselves, are soft and susceptible to deformation and damage if they are stored badly. Poorly stored tampons can absorb moisture, for example during intense rain or if a poorly sealed drink gets spilled, resulting in the destruction of the tampon. Poor storage of the tampon can lead to its contamination, clearly evident on the material, which is usually white.

Fashion and the general accustomed methods by which women dress has led to the fact that women store all their needs in handbags, bags, purses, and so on. To simplify, the entire range of handy aids for carrying things and the items that a woman needs to have with her is included in the word “handbag”. The disadvantages of handbags lie in the fact that women use them to carry all the belongings they need to have with them. If these belongings include spare menstrual tampons, they may become damaged due to the aforementioned deficiencies. Menstrual tampons must then be thrown away, or, in very uncomfortable extreme situations, must be applied.

Tampon manufacturers try to solve these problems by wrapping tampons into plastic or painted cellulose casing, or perhaps the woman may take the entire package of menstrual tampons in the cardboard sales box into her handbag. The disadvantages of this solution consist in the fact that the plastic or other packaging protects the tampons from moisture and dirt, but it does not protect tampons against mechanical damage, e.g. in the event where the tampon is damaged by a full drinking bottle located among the items in the purse. In most cases, cardboard sales boxes are subject to mechanical damage; tampons can spill all over the handbag, or even out of it. Simply gathering the damaged tampons that have spilled from the sales box into the handbag is bothersome work for women.

One of the most significant disadvantages of the aforementioned designs is that tampons are easily identifiable, especially the sales boxes. If a woman is digging through her handbag in the company of other people, the tampons could appear to other people in a conspicuous place, or they could spill out. This moment could be very annoying or almost humiliating for some women in some situations. Sales packages do not provide discretion. In the event that this situation happens to a woman in public, she is exposed to emotional pressure which could lead to other unpleasant events.

The invention aims to create a case for tampons which would remedy all the above disadvantages, which would be resistant to mechanical influences, which would be resistant to factors of contamination and moisture enough to protect the tampons, and which would allow women to store tampons discreetly, e.g. in their handbags.

### SUMMARY OF THE INVENTION

This objective is achieved by creating a case for menstrual tampons in accordance with the present invention.

The essence of the invention consists in that the tampon case is formed by a hollow body having a rigid housing and a top and bottom end opposite each other. The bottom end is closed and inside the housing there is arranged at least one dispensing means pushing towards the top. The top end is open and is provided with at least one means for holding the menstrual tampon in the top area. In the body of the case, the menstrual tampons are arranged in parallel in a single row between the dispensing means and the open top, while the dispensing means abuts and pushes on the edge of the single row of the tampons arranged in parallel.

The rigidity of the body adequately protects the tampons from the effects of external mechanical influences while at the same time prevents the penetration of dirt into the interior of the body and protects it from moisture. The bottom end forms a solid base upon which the dispensing means rests. This produces a force for pushing towards the top. The top end is open to allow passage of the tampons, but so that the row of tampons is not pushed out all at once, the top end is provided with at least one means for holding the tampon at the very top. The holder captures the tampon at the top and does not allow it to be pushed away by the other tampons waiting in the row. The tampon is removed from the top by the woman, while the next tampon waiting in the row takes its place due to the force of the dispensing means.

In a preferred embodiment of the case according to the invention, the dispensing means comprises at least one spring and at least one pressure base, whereby the spring is arranged between the bottom and the pressure base. Springs are inexpensive, durable, and are almost unbreakable when used properly. The compression spring tends to return to its original shape, while the force it exerts is easily selected by choosing the proper spring. The pressure base varies the strength from the end of the spring to a slight pressure, since it exerts through a continuous surface on the outer tampon of the row. The tampons are not chafed or deformed.

In another preferred embodiment of the case according to the invention, on the inner side of the case there is at least one guide ridge and at least one guide groove on the pressure base, wherein the guide ridge is located in the guide groove. In order to keep the bottom and the pressure base constantly horizontal to each other, it is appropriate to model a guide ridge into the inner housing that fits into the guide groove on the pressure base. No tilting occurs which could cause the spring to pass into the area above the pressure base, thus breaking the case.

In another preferred embodiment of the case according to the invention, the means for holding the tampon partially closes the top and simultaneously extends over the top, while exhibiting at least one abutment surface for abutting at least part of the cylindrical shell of the tampon. Partial closure of the top end prevents the row of stored tampons from escaping while keeping the function of filling the case from the top. The upper tampon, by extending over the top end, can be easily grasped between the fingers. The tampons are stopped by the abutment of the upper tampon against the abutment surface that compensates for the effect of the dispensing means.

In another preferred embodiment of the case according to the invention, the means for holding the tampon extends over the top to a height equal to 50% to 80% of the diameter of the tampon. The tampon, protruding half of its diameter or more, can be simply grabbed by the fingers while remaining slightly recessed into the case, thus preventing the tampon from arbitrary falling out in the direction of its cylindrical end.

In another preferred embodiment of the case according to the invention, the case is provided with a removable or hinged lid for covering the tampon held inside the top end. The lid closes the case completely to protect from negative influences that affect the quality of the tampons, to protect the tampon holder from damage, to conceal the tampons from unwanted eyes, and simultaneously completing the design of the case.

In another preferred embodiment of the case according to the invention, the case is made of at least one material from the group of plastic, wood, metal, or hard paper. These materials are well machinable, can be combined in many designs, and their surface can be changed. When properly processed, they also exhibit sufficient rigidity to protect the tampons from mechanical damage.

In another preferred embodiment of the case according to the invention, its outer surface is printable, or is adapted for gluing decorative items, or is carved or embossed. The case can be adjusted to a wide variety of appearances, collections, etc. The case thus obtains not only a practical, but also an aesthetic function, thanks to which it can suit any handbag.

In another preferred embodiment of the case according to the invention, the body of the case has rounded edges. The rounded edges can be very gently rounded for a pleasant tactile sensation while the ends retain their rectangular shape, or they may be very rounded giving the ends of the case an oval shape.

The tampon case is an aesthetic handbag accessory that prevents unpleasant situations while providing tampons with adequate protection from mechanical influences, contamination, and moisture. The case can be visually modified or provided with a variety of lids. The tampons can easily be found in the purse, completely eliminating the need to carry the entire sales package.

### CLARIFICATION OF THE DRAWINGS

The invention is more closely illustrated in the following drawings, wherein:

FIG. 1 shows a perspective view of the case without a lid,

FIG. 2 shows a partly transparent case with one spring,

FIG. 3 shows the case with a single spring in top view,

FIG. 4 shows a partly transparent case with two springs,

FIG. 5 shows the case with two springs from top view,

FIG. 6 shows a perspective view onto the case provided with a lid.

### EXAMPLES OF THE PREFERRED EMBODIMENTS OF THE INVENTION

It is understood that the hereinafter described and illustrated specific examples of the realization of the invention are presented for illustrative purposes and not as a limitation of the examples of the realization of the invention to the cases shown herein. Experts who are familiar with the state of technology shall find, or using routine experimentation will be able to determine, a greater or lesser number of equivalents to the specific realizations of the invention which are specifically described here. These equivalents shall also be included into the scope of the patent claims.

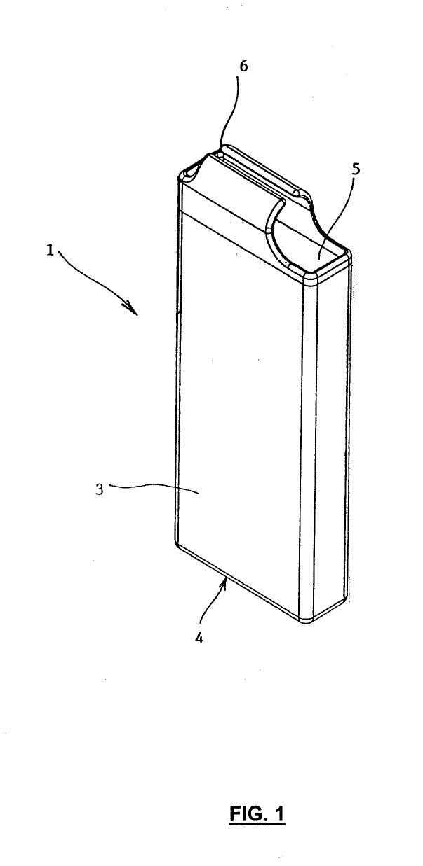

FIG. 1 shows the case 1 without a lid 12. The case 1 is block-shaped with a bottom end 4 and top end 5 having a rectangular shape with rounded corners. The housing 3 of the body follows the curvature of the corners of the bottom and top ends 4 and 5. At the open top end 5 there is a holder 6 for retaining the tampon 2 in its area. The case 1 is made of plastic with dimensions of height, including the holder 6 for retaining the tampon 2, of 117.5 mm, width of 45 mm, and depth of 16 mm.

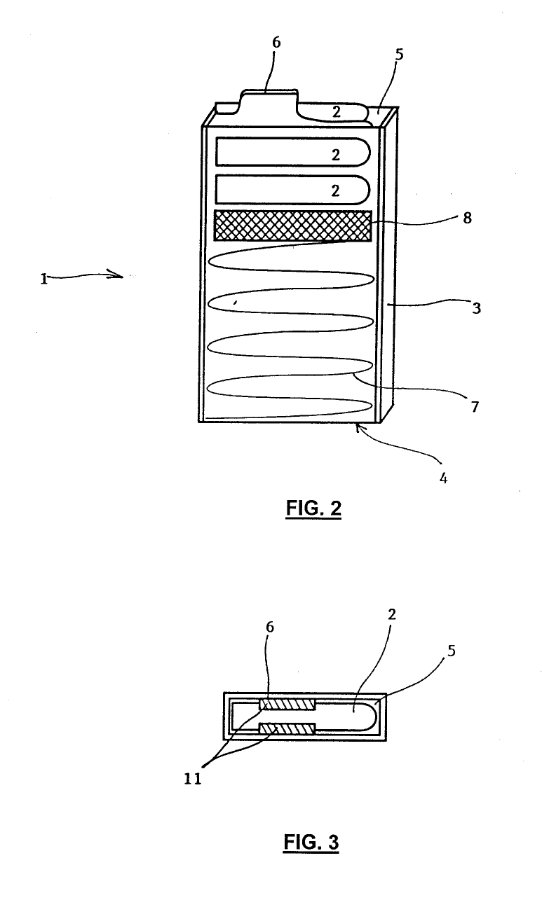

FIG. 2 depicts the case 1 with transparent part of the housing 3. Inside the case 1 there is depicted a row of tampons 2 with three pieces. The tampons 2 are extruded by the dispensing means, which consists of a spring 7 pressing against the bottom end 4 and the pressure base 8 pressing against the last tampon 2 of the row. The holder 6 for retaining the tampon 2 at the top end 5 prevents the entire row of tampons 2 from being pushed out all at once.

The holder 6 partly closes the top end 5 so that the tampons 2 are not pushed out all at once. The first tampon 2 of the row is stopped by the abutment surface H, which is highlighted in FIG. 3 for better illustration. The tampon 2 can only be drawn out by pulling in the direction of its cylindrical base. The case 1 is filled similarly, but in the opposite sense. The tampon 2 is inserted into the top end 5 below the holder 6, then is pressed against the abutment surfaces 11 and is fixed in place. When inserting the next tampon 2, the fixed tampon 2 is forced deeper into the case 1 (the force of the spring 7 is overcome) and the new tampon 2 is inserted into the row above it.

In another design (not shown), the tampons 2 need not be only in one row above each other. If the case 1 is wider in the area between the top end 5 and bottom end 4, an alternating double row of tampons 2 can be packed into the case 1, wherein the tampons 2 are arranged in one row at the top end 5.

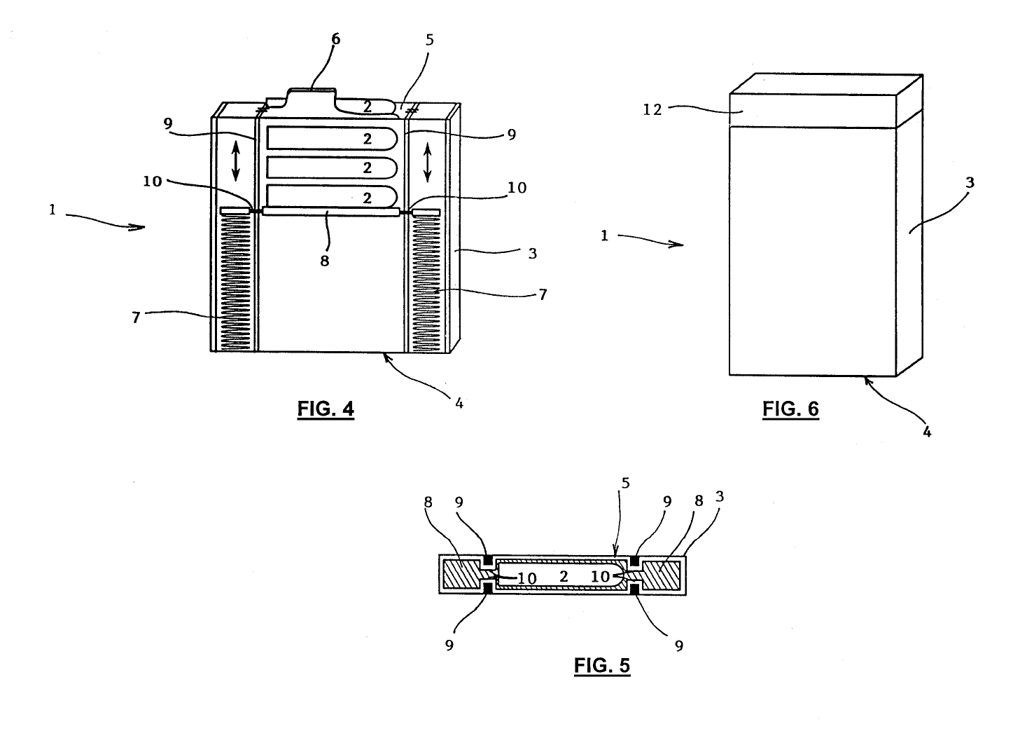

FIG. 4 shows the case 1 with a partially transparent housing 3 which is provided with two oppositely arranged springs 7 loaded under the pressure base 8. To prevent the pressure base 8 from diverting from the horizontal plane, it is provided with guide grooves 10 into which the guide ridges 9 engage. The guide ridges 9 are located on the inner side of the housing 3. A more detailed view of an embodiment of the pressure base 8, the guide grooves 10, and the guide ridges 9 is shown in FIG. 5, where the tampon holder 6 is not shown. The guide ridges 9 also form an obstacle that prevents the tampons 2 from sliding inside the case 1.

FIG. 6 shows the case 1 with a removable lid 12. The lid 12 is made of the same material as the housing of the case 1.

In examples not shown here, the case 1 may be decorated with stickers, decorative sticky paper, or can be printed directly. In the case of wood or plastic material, the case 1 may be carved or embossed. The ends 4 and 5 may have a different shape than a rectangle, provided that the function of dispensing means and the tampon holder 6 remain respected. An oval shape, for example, may be considered.

### INDUSTRIAL APPLICABILITY

The menstrual tampon case according to the invention will find application especially in handbags, luggage, and in the pockets of women who need to carry tampons with them.

### OVERVIEW OF THE POSITIONS USED IN THE DRAWINGS

1 case
2 menstrual tampon
3 hard housing
4 bottom end
5 top end
6 tampon holder
7 spring
8 pressure base
9 guide ridge
10 guide groove
11 supporting surface
12 lid

1. A case (1) for menstrual tampons (2), characterized in that it consists of a hollow body having a rigid housing (3) and two opposite ends (4, 5), the bottom end (4) of which is closed and towards which, inside the body, there is arranged at least one dispensing means acting towards the opposite end (5), the top end (5) is open and is provided with at least one holder (6) for retaining the menstrual tampon (2) in the top end (5), wherein the tampons (2) in the body of the case (1) are arranged parallel to one another into at least a single row and are in the area between the dispensing means and the open top end (5), wherein the dispensing means acts on the adjacent edge of the single row of parallel menstrual tampons (2).

2. A case according to claim 1, characterized in that the dispensing means consists of at least one spring (7) and at least one pressure base (8), wherein the spring (7) is arranged between the lower end (4) and the pressure base (8).

3. A case according to claim 2, characterized in that on the inner side of the housing (3) there is formed at least one guide ridge (9) while on the pressure base (8) there is formed at least one guide groove (10), wherein the guide ridge (9) fits into the guide groove (10).

4. A case according to claim 1, characterized in that the holder (6) for retaining the menstrual tampon (2) partially closes the top end (5) while extending above it, and that it also consists of at least one abutment surface (11) for abutting the menstrual tampon (2).

5. A case according to claim 4, characterized in that the holder (6) for retaining the menstrual tampon (2) extends above the top end (5) to a height corresponding to the size of 50% to 80% of the diameter of the menstrual tampon (2).

6. A case according to claim 1, characterized in that it is provided with a removable or hinged lid (12) for covering the menstrual tampon (2) held in the top end (5).

7. A case according to claim 1, characterized in that it is made of at least one material from the group of plastic, wood, metal, or hard paper.

8. A case according to claim 1, characterized in that its outer surface is printable, or is adapted for gluing decorative items, or is carved or embossed.

9. A case according to claim 1, characterized in that the body of the case (1) has rounded edges.
